(19) United States
(12) Patent Application Publication
(10) Pub. No.: US 2006/0200171 A1
(43) Pub. Date: Sep. 7, 2006

(54) Medical retrieval device and related methods of use

(75) Inventor: James A. Teague, Spencer, IN (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: BOSTON SCIENTIFIC SCIMED, Inc.

(21) Appl. No.: 11/071,438

(22) Filed: Mar. 4, 2005

(51) Int. Cl. A61B 17/22

(52) U.S. Cl. 606/127

(57) ABSTRACT

Embodiments of the invention are directed to a medical device for extracting material from a patient's body. The device may include a core element including a proximal portion extending substantially longitudinally and a distal portion capable of transforming between a coil configuration and a substantially straight configuration. An outer housing having a distal portion enclosing the distal portion of the core element is capable of attaining the coil and substantially straight configurations of the distal portion of the core element. The distal portions of the outer housing and the core element transform between the coil configuration and the substantially straight configuration when the core element is moved relative to the outer housing.

### DESCRIPTION OF THE INVENTION

1. Field of the Invention

This invention relates to medical devices for medical treatment of objects within anatomical lumens of the body, and more specifically to devices and methods for improving the manipulation of medical devices and the manipulation of objects treated within an anatomical lumen during a medical procedure.

2. Background of the Invention

Medical retrieval devices may include devices for treating and/or removing organic material (e.g., blood clots, tissue, and biological concretions such as urinary, biliary, and pancreatic stones) and inorganic material (e.g., components of a medical device or other foreign matter), which may obstruct or otherwise be present within a body's anatomical lumens. For example, concretions can develop in certain parts of the body, such as in the kidneys, pancreas, and gallbladder. Minimally invasive medical procedures generally involve causing limited trauma to the tissues of a patient, and can be used to dispose of problematic concretions. Lithotripsy and ureteroscopy, for example, are used to treat urinary calculi (e.g., kidney stones) in the ureter of patients.

Lithotripsy is a medical procedure that uses energy in various forms such as acoustic shock waves, pneumatic pulsation, electrical hydraulic shock waves, or laser beams to break up biological concretions such as urinary calculi (e.g., kidney stones). The force of the energy, when applied either extracorporeally or intracorporeally, usually in focused and continuous or successive bursts, divides a kidney stone into smaller fragments that may be extracted from the body or allowed to pass through urination. With the help of imaging tools such as transureteroscopic video technology and fluoroscopic imaging, the operator of the lithotripter device can monitor the progress of the medical procedure and terminate treatment when residual fragments are small enough to be voided or removed.

Intracorporeal fragmentation of urinary calculi can prove problematic in that stones and/or stone fragments in the ureter may become repositioned closer to and possibly migrate back toward the kidney, thereby requiring further medical intervention to prevent the aggravation of the patient's condition. It is desirable to be able to extract such fragments from the body using a single instrument, to prevent the need for successive instrumentation which can cause trauma to the lining of a patient's ureter.

Many known stone extraction devices are rigid and lack the maneuverability and flexibility to engage and disengage repeatedly a stone without harming the surrounding tissue. For example, if a stone is still too large to be extracted without further fragmentation, it can be difficult to disengage the stone from such an extraction device without damaging the delicate lining of the ureteral wall.

Various coiled medical extraction devices are known. For example, referring to FIG. 1, a known embodiment of a coiled medical extraction device 10 includes a sheath 12 and a core element 14. The embodiment of FIG. 1 is also disclosed in U.S. Pat. No. 6,740,096, issued on May 25, 2004, which is hereby incorporated by reference in its entirety. The core element 14 can be made at least partially of a shape-memory material. Shape-memory material is a material that can be formed into a particular shape, retain that shape during resting conditions (e.g., when the shaped material is in free space or when external forces applied to the shaped material are insufficient to substantially deform the shape), be deformed into a second shape when subjected to a sufficiently strong external force, and revert substantially back to the initial shape when external forces are no longer applied. Examples of shape memory materials include synthetic plastics, stainless steel, and superelastic, metallic alloys of nickel/titanium (commonly referred to as nitinol), copper, cobalt, vanadium, chromium, iron, or the like.

As seen in FIG. 1, the core element 14 includes a proximal portion 18, which extends substantially longitudinally, and a distal portion wound to form a helical coil 16 in the absence of external forces. The helical coil 16 is adapted to taper from a larger diameter at a proximal end thereof to a smaller diameter at a distal end thereof, thereby resembling a helical cone shape. The sheath 12 and core element 14 are movable relative to each other in order to achieve a first, substantially linear, collapsed state (not shown) in which the distal portion of the core element 14 is collapsed within the lumen of the sheath 12 and a second state in which the distal portion of the core element 14 extends from the distal end of the sheath 12 and expands to form a helical coil 16 (seen in FIG. 1). Known coiled medical extraction devices can include a polymer coating along all or part of the core element 14 for reducing the amount of friction between the surfaces of core element 14 and sheath 12 during movement between expanded and collapsed states. In addition, the polymer coating is intended to reduce friction between the core element 14 and the lining of the ureteral wall into which the device is deployed.

Coiled medical extraction devices, like that shown in FIG. 1, can be used to prevent the upward migration of stone fragments generated during a stone fragmentation procedure, and then safely and efficiently extract fragments from the body. For example, during a lithotripsy procedure, a coiled medical extraction device can act as a backstop against any upward migration of stone fragments resulting from the procedure. The extraction device may be then used to remove the fragments from the body. Coiled medical extraction devices further enable repeated application to stones, stone fragments, and other biological and nonbiological/foreign material following consecutive lithotripsy procedures.

If a stone is still too large to be extracted without further fragmentation or an obstacle is encountered upon forward movement of the material within the anatomical lumen, coiled medical extraction devices, like that shown in FIG. 1, have the capability to releasably disengage the stone by, for example, obtaining a straightened, non-coiled state. The coil can attain a substantially straightened shape and unwind upon retraction of the core element within the sheath 12. When an oversized stone is ensnared within the coiled medical extraction device, however, there may be little room, if any, between the ensnared stone and the ureteral wall. Accordingly, the retraction and unwinding of the core element from the position of ensnaring an oversized stone to a collapsed state within a sheath can create friction that is potentially abrasive to the internal tissue of the anatomical lumen.

Coiled medical extraction devices, like the device of FIG. 1, can include a sheath (e.g. sheath 12) that is advanced over the coiled section in order to impart a straightened, non-coiled shape to the device for delivering a straightened section to a body location, disengaging a retrieved material by unwinding the coiled shape, and removing the device from within an anatomical lumen of the patient upon attaining the straightened shape. As coils are strengthened in order to increase the force of retrieval, stronger and larger over-sheaths are used to straighten the coiled section. As a result, in some situations, deployment of a large over-sheath may be inhibited by restrictive anatomy or undesired biological and foreign materials in the anatomical lumen.

In addition, the deployment of a large over-sheath for straightening will increase the overall outer diameter of the medical retrieval system. This increased size can create problems for an operator by limiting, for example, the ability to irrigate around the retrieval device via the auxiliary channel of an endoscope through which the retrieval device is often deployed in a patient's body.

In light of the foregoing, there is a need for an improved coiled medical extraction device that allows for the deployment and redeployment between straightened and coiled shapes without the need for a conventional over-sheath.

### SUMMARY OF THE INVENTION

The present invention is directed to a medical device for extracting material from a patient's body that obviates one or more of the limitations and disadvantages of the prior art medical extraction devices.

In one embodiment, the medical device includes a core element including a proximal portion extending substantially longitudinally and a distal portion capable of transforming between a coil configuration and a substantially straight configuration. An outer housing having a distal portion enclosing the distal portion of the core element is capable of attaining the coil and substantially straight configurations of the distal portion of the core element. The distal portions of the outer housing and the core element transform between the coil configuration and the substantially straight configuration when the core element is moved relative to the outer housing.

In various embodiments, the medical device may include one or more of the following additional features: a distal tip of the core element extends outside the outer housing; the distal tip provides a compressive force to the outer housing to achieve the substantially straight configuration; wherein the distal portions of the outer housing and the core element attain the substantially straight configuration upon proximal movement of the core element relative to the outer housing; wherein the distal portions of the outer housing and the core element attain the coil configuration upon distal movement of the core element relative to the outer housing; wherein the core element comprises a shape-memory material; wherein the coil configuration is adapted to ensnare objects in an anatomical lumen; wherein the distal tip of the core element includes a retaining element having a diameter greater than an inner diameter of the outer housing; a handle connected to a proximal end of the outer housing and a proximal end of the core element for providing relative movement between the core element and the outer housing; wherein the handle includes a first piece connected to a proximal end of the core element and a second piece connected to a proximal end of the outer housing and the first and second piece are engaged for relative movement between the two pieces upon actuation of the handle; wherein rotation of the second piece relative to the first piece results in longitudinal movement of second piece relative to the first piece; wherein the distal portion of the outer housing includes a series of interconnected discrete segments; wherein adjacent discrete segments are connected by linked engagement; wherein each discrete segment includes a protruding portion on one end and a receiving portion on another end, and adjacent discrete segments are linked by the engagement of respective protruding portions and receiving portions; wherein adjacent discrete segments are pivotally movable relative to each other; wherein a proximal portion of the outer housing includes, a flexible cannula extending proximally from the series of interconnected discrete segments; and wherein proximal movement of the core element relative to the outer housing generates a compressive force along the outer housing that straightens the distal portion of inner core.

Another embodiment of the invention is directed to a method for retrieving material in a body. The method includes providing a medical device including a core element a proximal portion extending substantially longitudinally and a distal portion capable of transforming between a coil configuration and a substantially straight configuration. The device includes an outer housing having a distal portion enclosing the distal portion of the core element and capable of attaining the coil and substantially straight configurations of the distal portion of the core element. The method further comprises inserting the medical device in the substantially straight configuration into an anatomical lumen of the body; positioning the distal portions of the core element and the outer housing beyond the material to be retrieved within the lumen; transforming the distal portions of the core element and the outer housing to the coil configuration; and retrieving the material with the distal portions of the outer housing and core element.

In various embodiments, the method may include one or more of the following additional features: pulling the medical device proximally when the material is retrieved with the distal portions of the outer housing and core element; performing a lithotripsy procedure on the material; transforming the medical device from the substantially straight configuration to an intermediate configuration between the substantially straight configuration and the coil configuration; wherein the core element exerts a first compressive force on the outer housing when the distal portions of the core element and the outer housing are in the substantially straight configuration, exerts a second compressive force less than the first compressive force on the outer housing when the distal portions of the core element and the outer housing are in the intermediate configuration, and exerts a third compressive force less than the second compressive force on the outer housing when the portions of the core element and the outer housing are in the coil configuration; wherein the third compressive force is substantially zero force; and after retrieving the material, transforming the distal portions of the core element and the outer housing to the substantially straight configuration; wherein a distal tip of the core element extends outside the outer housing; wherein the distal tip of the core element includes a retaining element having a diameter greater than an inner diameter of the outer housing; wherein the distal portion of the outer housing includes a series of interconnected discrete segments; wherein transforming the distal portions of the core element and outer housing to the coil configuration includes pivoting a discrete segment relative to an adjacent discrete segment; a handle connected to a proximal end of the outer housing and a proximal end of the core element for providing relative movement between the core element and the outer housing; wherein the handle includes a first piece connected to a proximal end of the core element and a second piece connected to a proximal end of the outer housing, and the first and second piece are engaged for relative movement therebetween upon actuation of the handle; wherein transforming the distal portions of the core element and the outer housing includes rotating the second piece relative to the first piece; and wherein rotating the second piece relative to the first piece controls an amount of compressive force the core element exerts on the outer housing.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments of the invention and together with the description, serve to explain the principles of the invention.

FIG. 1 is a side view of a coiled medical extraction device in a deployed position.

FIG. 2 is a distal portion of a coiled medical extraction device in a deployed configuration, according to an embodiment of the invention.

FIG. 3 is a partial side cross-section view of the distal portion of the coiled medical extraction device of FIG. 2 in a straightened configuration.

FIG. 4A is a partial side cross-sectional view of a handle mechanism for a coiled medical extraction device, according to an embodiment of the present invention, in a straightened configuration.

FIG. 4B is a partial side cross-sectional view of a handle mechanism for the coiled medical extraction device of FIG. 4A, in a deployed configuration.

FIG. 5 is a side cross-sectional view of an alternative handle mechanism for a coiled medical extraction device, according to an embodiment of the present invention.

### DESCRIPTION OF THE EMBODIMENTS

Reference will now be made in detail to the present exemplary embodiments of the invention illustrated in the accompanying drawings. Wherever possible, the same reference numbers will be used throughout the drawings to refer to the same or like parts.

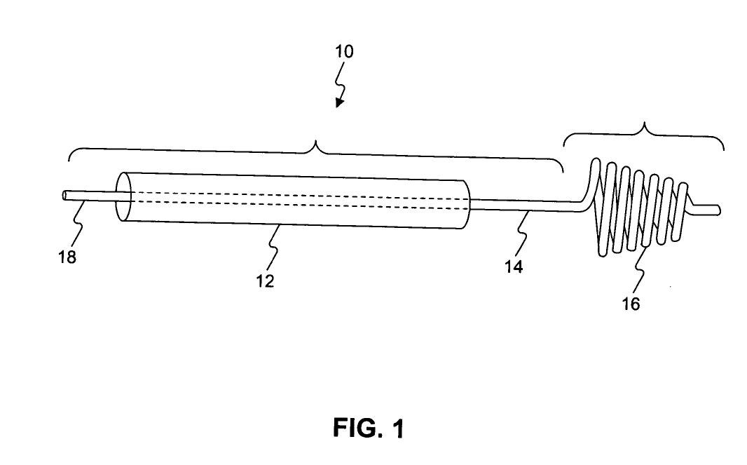

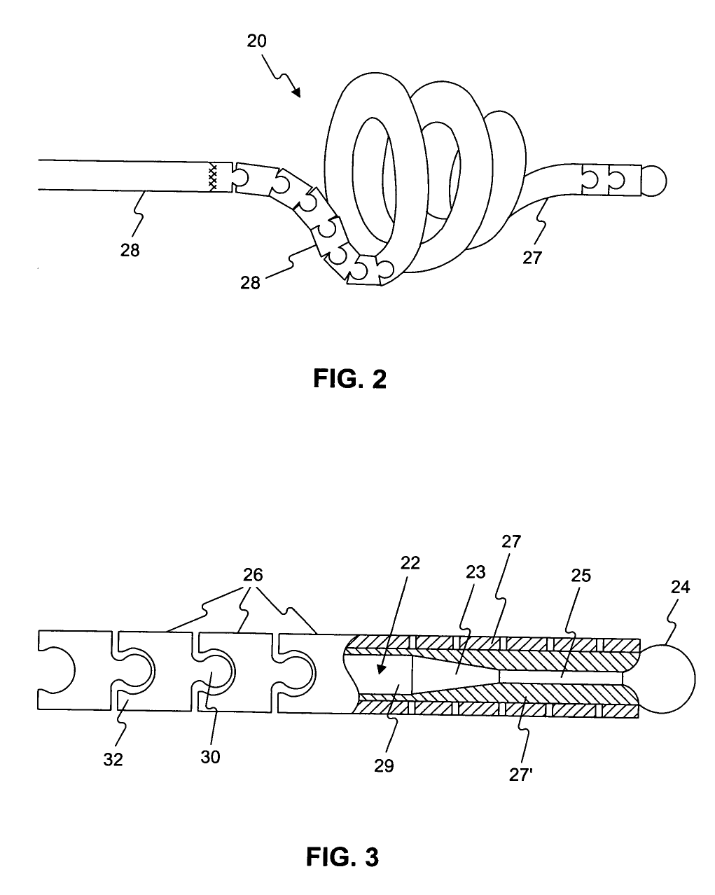

Embodiments of the invention relate to coiled medical extraction devices that do not include a conventional over-sheath for actuating the distal portion of a core element between a coiled state and a substantially straight state. For example, FIG. 2 illustrates a coiled medical extraction device 20, according to an embodiment of the invention. Medical extraction device 20 is capable of actuated deployment between a coiled state (shown in FIG. 2) and a substantially linear state (shown in FIG. 3) without the necessity of an external sheath 12 as in the example of FIG. 1. The medical extraction device 20 includes an inner core element 22 (see FIG. 3) and outer housing 27.

Core element 22 can be formed in part of a shape memory material, such as synthetic plastic, stainless steel, superelastic, metallic alloys of nickel/titanium (commonly referred to as nitinol), copper, cobalt, vanadium, chromium, iron, or the like. The distal portion of inner core element 22 attains a coiled state when unrestrained by external forces. As seen in FIG. 3, the inner core element 22 includes an elongate proximal portion 29, a tapered section 23, a reduced diameter portion 25, and a distal tip formed of a ball 24. Proximal portion 29 may extend proximally to a handle mechanism and has a larger diameter than section 23 and portion 25. The distal portion of element 22 that forms the coiled state may include at least a part of proximal portion 29. That distal portion can be pre-formed, either by use of shape memory material or other suitable known materials and methods, to naturally attain the shape of a helical coil when unrestrained by external forces. Tapered section 23 and reduced diameter portion 25 provide increased flexibility at the distal most portion of the inner core element 22 for reducing potential trauma to a patient's tissues upon movement and repeated actuation of medical extraction device 20. Ball 24 is a retaining element having a diameter greater than that of the remaining portion of the inner core element 22.

Medical extraction device 20 includes an outer housing 27 having an internal lumen 27′ which receives the inner core element 22, except for the ball 24 at the distal tip. The outer housing 27 includes a proximal portion comprised of a flexible outer cannula 28 and a distal portion comprised of a series of interconnected flexible discrete segments 26. The flexible outer cannula 28 can be manufactured out of materials such as stainless steel, cobalt chromium, a chrome doped nickel-titanium alloy, a nickel-titanium alloy, or other suitable materials. The interconnected flexible discrete segments 26 can be formed through a process of laser cutting or chemically etching the same material forming the outer cannula 28. The distal end of the outer cannula 28 can be laser welded to the proximal most discrete segment 26 for forming the outer housing 27. Alternatively, cannula 28 and segments 26 may be integrally formed.

Interconnected flexible discrete segments 26 enclose the distal portion of core element 22 that forms the helical coil. The series of discrete segments 26 are connected by cooperative linked engagement between adjacent discrete segments 26. Each segment 26 may include a convex protruding portion 30 on one end, and a concave receiving portion 32 on another end. Adjacent segments 26 are cooperatively linked by the engagement of adjacent convex protruding portions 30 and concave receiving portions 32. In addition, this linked engagement between discrete segments 26 permits pivoting movement of segments 26 relative to each other. FIG. 2 shows segments 26 pivoted relative to one another, permitting the distal portion of inner core 22 to obtain the helical coil shape. It is contemplated that adjacent discrete members could be connected or engaged by suitable alternative designs, other than the illustrated concave and convex portions, so long as the members remain engaged and movable relative to one another.

Referring to FIG. 3, the diameter of the retaining ball 24 is also greater than the inner diameter of the distal most discrete segment 26 such that the distal retaining ball 24 cannot be moved proximal to the discrete segments 26. In FIG. 3, the space and relative pivoting between adjacent discrete segments 26 has been reduced, for example by distal movement of outer housing 27 relative to the inner core 22. The relative movement between housing 27 and core 22 causes ball 24 to exert a force against the distal most segment 26. This force compresses segments 26 to form a substantially straight arrangement. This also causes the distal portion of inner core 22 to transform from the helical shape to a substantially straight configuration. Accordingly, the medical extraction device 20 is capable of actuated deployment between coiled and substantially linear states by actuated tightening of the interconnected flexible discrete segments 26 by relative movement between the inner core element 22 and the outer housing 27.

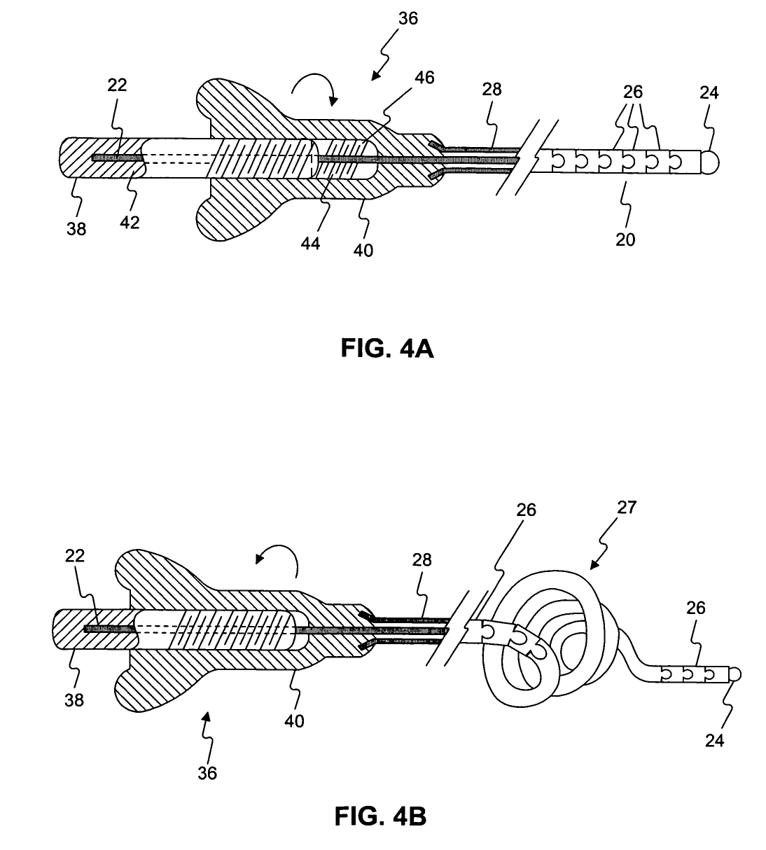

Various proximal handle mechanisms may be used that cause relative movement between core element 22 and housing 27. For example, FIGS. 4A and 4B illustrate a handle 36 for a coiled medical extraction device 20. Handle 36 includes an inner handle piece 38 connected to an outer handle piece 40. Inner handle piece 38 may include exterior threads 42 inter-engaged with corresponding thread grooves 44 along an interior surface of the outer handle piece 40. Outer handle piece 40 defines a hollow space 46 that receives handle piece 38. The inner core element 22 may be fastened at its proximal end to the inner handle piece 38, while outer handle piece 40 is connected at its distal end to the proximal end of the flexible outer cannula 28.

As seen in FIG. 4A, when the outer handle piece 40 is rotated clockwise relative to the inner handle piece 38 (viewing from the proximal end), the outer housing 27 is advanced forward relative to the inner core element 22. This relative movement in turn results in a longitudinally directed compressive force created by the retaining ball 24 against the distal most discrete segment 26. As described above, the compressive force reduces the space and relative pivoted state between adjacent segments 26 such that the resulting tightening force straightens the distal portion of inner core 22.

FIG. 4B illustrates the coiled medical extraction device 20 in its relaxed coiled state. When the outer handle piece 40 is rotated counter-clockwise (viewing from the proximal end) relative to the inner handle piece 38, the relative movement between housing 27 and core element 22 reduces, or may entirely remove, any compressive force generated by retaining ball 24 against the distal most discrete segment 26. As noted above, in a loose rest position of the medical device 20, the discrete segments 26 are pivotally movable relative to each other and are thereby free to obtain the helical coil shape imparted by the distal portion of the inner core element 22. In the position of FIG. 4B, the relaxed coiled shape along the distal portion of the inner core element 22 is unhindered by any compressive or tightening force imparted by relative movement between the inner core 22 and the outer housing 27.

A number of factors can limit the size and/or shape of the coiled state of the device or can affect the degree to which an operator controls the transition of the coiled medical extraction device 20 from the configuration of FIG. 4A to the configuration of FIG. 4B. For example, as noted above, the relative movement between the inner core element 22 and the outer housing 27 causes the straightening of the distal portion of inner core element 22. This relative movement may be limited by the distance available for inner handle piece 38 to move within hollow space 46 of the outer handle piece 40. For example, should the distal most end of inner handle piece 38 contact the distal most surface defining the hollow space 46, prior to the complete transformation of the relaxed coiled shape, an intermediate configuration could be purposefully attained. The expanded state of such an intermediate configuration could allow an operator to limit the extent to which the inner core element reaches the fully coiled configuration. Preferably, the range of motion does not restrain the desired end shape of the expanded coiled configuration.

Another factor that may limit the size and/or shape of the coiled state of the device may include the range of relative pivotal movement between adjacent discrete segments 26. In the relaxed coiled configuration of FIG. 4B, the discrete segments 26 preferably are able to pivot sufficiently relative to each other such that the outer housing 27 does not restrain the desired end shape of the expanded coiled configuration imparted by inner core element 22.

The pitch size of threads associated with the handle pieces 38, 40 may aid in controlling the transition between the substantially straight and coiled configurations. The greater the pitch to the threads 42 and grooves 44 of the engaged handle pieces 38, 40, the greater the number of rotations necessary to attain a complete transition from the state of FIG. 4A to the state of FIG. 4B. This arrangement may allow for precise adjustment between the two states and variance in the amount of compressive force that ball 24 exerts on distal segments 26. Embodiments of the present invention thereby allow the operator to more carefully control the state of expansion of coiled medical extraction device 20. Increased control by an operator can allow adjustment of the expansion as desired depending upon such environmental factors as the size of the patient's internal body lumen and the size of kidney stones or fragments encountered.

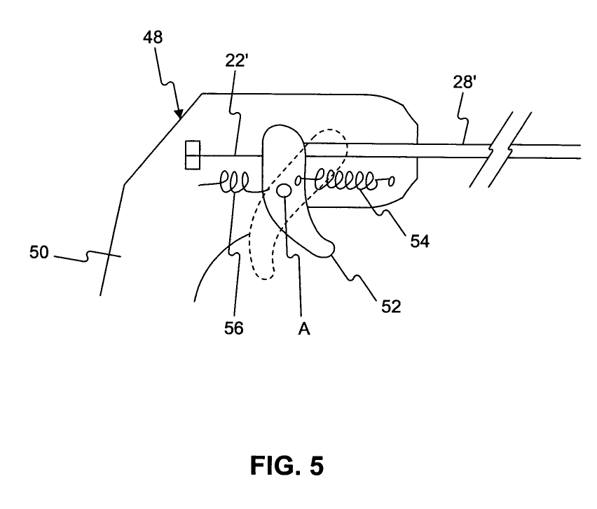

FIG. 5 illustrates an alternative handle 48 for a coiled medical extraction device 20. Handle 48 includes a pistol grip body 50 and a trigger 52 for actuating the deployment of the coiled medical extraction device 20 between coiled and straightened shapes. The trigger 52 pivots about point A and can be operatively engaged with the proximal most end of the proximal outer cannula 28′. The proximal end of the inner core element 22′ is internally connected to an interior point within the pistol grip body 50 of handle 48.

In addition, trigger 52 can be spring loaded by springs 54 and 56. Springs 54 and 56 may bias trigger 52 into a position corresponding to either a straight shape or a coiled shape of the distal portion of the medical extraction device 20. In other words, the configuration of handle 48 may be set such that the coiled medical extraction device 20 attains the coiled shape upon actuation or alternatively may be set such that the coiled medical extraction device 20 attains the substantially straightened shape upon actuation.

For example, as shown in FIG. 5, the solid lines for trigger 52 may represent an at rest position caused by biasing springs 54, 56 and the dashed lines for trigger 52 may represent an actuated position of trigger 52 against the bias of springs 54, 56. Upon actuation of the trigger 52, the proximal outer cannula 28′ is advanced forward relative to the proximal end of inner core element 22′ connected within the handle 48. This relative movement in turn results in a longitudinally directed compressive force created by the abutment of the retaining ball 24 (not shown) against the distal most discrete segment 26 (not shown). The compressive force reduces the space between adjacent segments 26 such that the resulting tightening force straightens the distal portion of inner core 22′.

The operation of the coiled medical extraction device 20 will now be described. In use, an operator inserts the coiled medical extraction device 20, with the helical coil in its substantially linear configuration (as shown in FIG. 4A), into an anatomical lumen until the distal portion of the inner core element 22 is positioned beyond an object in the anatomical lumen. For example, in a lithotripsy procedure to remove a kidney stone from a patient's ureter, the coiled medical extraction device 20 is introduced into the patient's urinary passage until the distal portion of the inner core element 22 passes beyond the location of a stone lodged in the ureter. A ureteroscope may be used for introduction of the device 20. The operator then transforms the coiled medical extraction device 20 to attain the helical coil shape (see FIG. 4B) in a manner as described above.

As the helical coil is released, the medical extraction device transforms into the helical cone configuration illustrated in FIG. 2 and may substantially occlude the anatomical lumen. The diameter of the helical coil may be sized to be substantially the same as or slightly greater than that of the anatomical lumen, so that the passage will be sufficiently occluded and prevent any subsequent migration of the kidney stone. Alternatively, the coil may expand to a diameter sufficient to ensnare the stone therein.

With the helical coil in its deployed position, the operator can pull the device proximally by means of handle 36 in order to ensnare the stone within the helical coil. At this point, a lithotripsy procedure may be performed to fragment the stone into smaller fragments. The helical coil serves as a physical barrier or back-stop during the lithotripsy procedure to ensure that the smaller fragments do not migrate in an undesired direction, e.g., kidney stone fragments migrating back toward the kidney. The superelasticity of the helical coil coupled with its conical configuration, provides a flexible barrier that is able to absorb the kinetic energy of the fragments produced when a laser or other energy is used to comminute or ablate the obstruction.

Once the lithotripsy procedure is complete, the operator pulls device 20 to ensnare the fragments. If the fragments are small enough to pass through the anatomical lumen, then the user can drag the fragments from the anatomical lumen and out of the body. However, if the fragments are still too large to pass through sections of the anatomical lumen, then the operator may straighten the coiled shape of the inner core element's distal portion, if disengagement of the trapped stone is desired. The operator can repeat the treatment procedure by redeploying the helical coil shape beyond the stone and performing a second lithotripsy procedure to further fragment the remaining obstructions.

In embodiments of the invention, the actuation between coiled and straightened shapes without the relative movement between a conventional over-sheath and the device, prevents additional trauma to the internal tissues of the patient's anatomical lumen. The outer diameter of extraction devices according to embodiments of the present invention can be reduced in comparison with other coiled medical extraction devices, in that the need for a large diameter sheath strong enough to unwind and collapse the coiled core element is removed. As noted above, prior coiled medical extraction devices can include a polymer coating along all or part of the core element for reducing the amount of friction between the surfaces of a core element and a conventional over-sheath during movement between expanded and collapsed states. Accordngly, the outer diameter of the extraction devices according to embodiments of the present invention can be further reduced because no additional polymer coating in necessary to reduce the friction of the core element.

For example, outer housing 27 may have an outer diameter of 0.038 inches or less, while prior coiled medical extraction devices have had outer diameters of about 0.043 inches. In embodiments of the present invention, the outer housing preferably remains in a position to enclose the inner core element 22 and its relatively smaller diameter reduces trauma to the patient by providing a reduced profile medical device.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device comprising:
a core element including a proximal portion extending substantially longitudinally and a distal portion capable of transforming between a coil configuration and a substantially straight configuration;
an outer housing having a distal portion enclosing the distal portion of the core element and capable of attaining the coil and substantially straight configurations of the distal portion of the core element; and
wherein the distal portions of the outer housing and the core element transform between the coil configuration and the substantially straight configuration when the core element is moved relative to the outer housing.

2. The medical device of claim 1 wherein a distal tip of the core element extends outside the outer housing.

3. The medical device of claim 2 wherein the distal tip provides a compressive force to the outer housing to achieve the substantially straight configuration.

4. The medical device of claim 1 wherein the distal portions of the outer housing and the core element attain the substantially straight configuration upon proximal movement of the core element relative to the outer housing.

5. The medical device of claim 1 wherein the distal portions of the outer housing and the core element attain the coil configuration upon distal movement of the core element relative to the outer housing.

6. The medical device of claim 1 wherein the core element comprises a shape-memory material.

7. The medical device of claim 1 wherein the coil configuration is adapted to ensnare an object in an anatomical lumen.

8. The medical device of claim 2 wherein the distal tip of the core element includes a retaining element having a diameter greater than an inner diameter of the outer housing.

9. The medical device of claim 1 further comprising a handle connected to a proximal end of the outer housing and a proximal end of the core element for providing relative movement between the core element and the outer housing.

10. The medical device of claim 9 wherein the handle includes a first piece connected to a proximal end of the core element and a second piece connected to a proximal end of the outer housing, and the first and second pieces are engaged for relative movement therebetween upon actuation of the handle.

11. The medical device of claim 10 wherein rotation of the second piece relative to the first piece results in longitudinal movement of second piece relative to the first piece.

12. The medical device of claim 1 wherein the distal portion of the outer housing includes a series of interconnected discrete segments.

13. The medical device of claim 12 wherein adjacent discrete segments are connected by linked engagement.

14. The medical device of claim 13 wherein each discrete segment includes a protruding portion on one end and a receiving portion on another end, and adjacent discrete segments are linked by the engagement of respective protruding portions and receiving portions.

15. The medical device of claim 12 wherein adjacent discrete segments are pivotally movable relative to each other.

16. The medical device of claim 12 wherein a proximal portion of the outer housing includes a flexible cannula extending proximally from the series of interconnected discrete segments.

17. The medical device of claim 1 wherein proximal movement of the core element relative to the outer housing generates a compressive force along the outer housing that substantially straightens the distal portion of the core element.

18. The medical device of claim 8 wherein the distal portion of the outer housing includes a series of interconnected discrete segments.

19. The medical device of claim 18 wherein adjacent discrete segments are connected by linked engagement.

20. The medical device of claim 19 wherein each discrete segment includes a protruding portion on one end and a receiving portion on another end, and adjacent discrete segments are linked by the engagement of respective protruding portions and receiving portions.

21. The medical device of claim 18 wherein adjacent discrete segments are pivotally movable relative to each other.

22. A method for retrieving material in a body comprising:
providing a medical device comprising:
a core element including a proximal portion extending substantially longitudinally and a distal portion capable of transforming between a coil configuration and a substantially straight configuration; and
an outer housing having a distal portion enclosing the distal portion of the core element and capable of attaining the coil and substantially straight configurations of the distal portion of the core element;
inserting the medical device into an anatomical lumen of the body, with the distal portions of the core element and the outer housing in the substantially straight configuration;
positioning the distal portions of the core element and the outer housing beyond the material to be retrieved within the lumen;
transforming the distal portions of the core element and the outer housing to the coil configuration by moving the core element relative to the outer housing; and
retrieving the material with the distal portions of the outer housing and core element.

23. The method of claim 22 further comprising pulling the medical device proximally when the material is retrieved with the distal portions of the outer housing and core element.

24. The method of claim 22 further comprising performing a lithotripsy procedure on the material.

25. The method of claim 22 further comprising transforming the medical device from the substantially straight configuration to an intermediate configuration between the substantially straight configuration and the coil configuration.

26. The method claim 25, wherein the core element exerts a first compressive force on the outer housing when the distal portions of the core element and the outer housing are in the substantially straight configuration, exerts a second compressive force less than the first compressive force on the outer housing when the distal portions of the core element and the outer housing are in the intermediate configuration, and exerts a third compressive force less than the second compressive force on the outer housing when the distal portions of the core element and the outer housing are in the coil configuration.

27. The method claim 26, wherein the third compressive force is substantially zero force.

28. The method of claim 22 further comprising, after retrieving the material, transforming the distal portions of the core element and the outer housing to the substantially straight configuration.

29. The method of claim 22 wherein a distal tip of the core element extends outside the outer housing.

30. The method of claim 29 wherein the distal tip of the core element includes a retaining element having a diameter greater than an inner diameter of the outer housing.

31. The method of claim 22 wherein the distal portion of the outer housing includes a series of interconnected discrete segments.

32. The method of claim 31, wherein transforming the distal portions of the core element and outer housing to the coil configuration includes pivoting a discrete segment relative to an adjacent discrete segment.

33. The method of claim 22 further comprising a handle connected to a proximal end of the outer housing and a proximal end of the core element for providing relative movement between the core element and the outer housing.

34. The method of claim 33 wherein the handle includes a first piece connected to a proximal end of the core element and a second piece connected to a proximal end of the outer housing, and the first and second piece are engaged for relative movement therebetween upon actuation of the handle.

35. The method of claim 34 wherein transforming the distal portions of the core element and the outer housing includes rotating the second piece relative to the first piece.

36. The method of claim 35 wherein rotating the second piece relative to the first piece controls an amount of compressive force the core element exerts on the outer housing.
